(19) United States
(12) Patent Application Publication
(10) Pub. No.: US 2011/0045517 A1
(43) Pub. Date: Feb. 24, 2011

(54) Toxic Material Detection Apparatus and Method

(75) Inventors: Tricia L Derringer, Lancaster, OH (US); Matthew J Shaw, Rockbridge, OH (US); Rodney S Black, Galloway, OH (US); Trevor Petrel, Columbus, OH (US); Fred Moore, Hilliard, OH (US); Laurence Slivon, Mt. Vernon, OH (US); David N Clark, Marysville, OH (US); Tom Danison, Grove City, OH (US)

Correspondence Address: DIEDERIKS & WHITELAW, PLC, 13885 HEDGEWOOD DR., SUITE 317, WOODBRIDGE, VA, 22193, US

(73) Assignee: BATTELLE MEMORIAL INSTITUTE, Columbus, OH (US)

(21) Appl. No.: 12/741,761

(22) PCT Filed: Nov. 6, 2008
(86) PCT No.: PCT/US08/82638

§ 371 Date: Oct. 8, 2010

Related U.S. Application Data
(60) Provisional application No. 60/985,843, filed on Nov. 6, 2007.

(51) Int. Cl. C12Q 1/46; C12M 1/34

(52) U.S. Cl. 435/20; 435/287.3

(57) ABSTRACT

A toxic material detection apparatus includes a sample collection portion including a sample inlet and a sample concentrator adapted to concentrate an environmental sample on a substrate. A sample distributing system transfers portions of the substrate to a color sensor and an ion mobility spectrometer for simultaneously analysis and toxin detection, particularly cholinesterase inhibitor detection. Optionally, a portion of the substrate may be directed to an archive for possible analysis at a later time. Reagents utilized include the enzyme acetylcholinesterase (AChE), and the reactants acetylthiocholine iodide (ATCI) and 5,5′-dithio-bis-(2-nitrobenzoic acid) (DTB). A data management unit provides for near-real-time analysis of the samples in under 5 minutes. Simultaneous “hits” by both analysis methods indicate the presence of a cholinesterase inhibitor.

### CROSS REFERENCE TO RELATED APPLICATION

This application claims priority of U.S. Provisional Patent Application No. 60/985,843 filed 6 Nov. 2007.

### BACKGROUND OF THE INVENTION

1. Field of the Invention

The present invention pertains to the art of toxic material detection and, more particularly, to the detection of neurotoxins.

2. Discussion of the Prior Art

Highly-toxic materials or HTMs are likely to be present as primary or secondary liquid or solid aerosols on or near battlefields and can pose a significant threat to human life. Of particular concern are cholinesterase inhibitors such as VX or Sarin gas. Biochemically, acetylcholinesterase (AChE) terminates nerve impulse transmissions at cholinergic synapses by hydrolyzing the neurotransmitter acetylcholine to acetate and choline. Cholinesterase inhibitors act by binding to AChE, which inhibits this vital enzyme's normal biological activity in the cholinerergic nervous system. The result is a build-up of acetylcholine, causing constant transmission of nerve signals. Even at very low concentrations, cholinesterase inhibitors can be fatal.

There are a number of methods for detecting specific toxic compounds in water and air, however, the methods suggested heretofore are either too slow to make them useful for real time detection, or are not easily transported to a location of interest. One proposed solution was offered by U.S. Pat. No. 7,422,892, which teaches an enzyme-based environmental monitoring device wherein a sample stream is continuously sampled and delivered to a butyryl cholinesterase carrying polyurethane polymer along with a substrate stream. As long as the sample is free of cholinesterase inhibitors, enzyme activity within the polymer decreases the pH of the substrate solution, causing a color change. The color change is monitored by a RGB (red, green, blue) color to frequency converter and control system. Despite this proposed solution, there is still seen to exist a need in the art for an improved field detector that can provide near real time detection of low volatility HTMs with high confidence.

### SUMMARY OF THE INVENTION

The present invention is directed to a toxic material detection apparatus and method. Among the materials to be detected by the present invention are low-volatility, cholinesterase inhibiting toxic materials. In general, the apparatus includes a sample concentrator or collector, a color sensor, an ion mobility spectrometer (IMS), a sample distributing system and a data manager and signal output. The sample collector concentrates and deposits aerosols, either in a dry or wet form, by impaction, filtration, or other suitable method, onto a substrate which may be a cloth, a membrane, or other suitable surface. Portions of the substrate are simultaneously directed to both the color sensor and IMS for analysis.

A heater is utilized to heat the deposited aerosol on a first substrate portion to form a vapor that is then introduced into the IMS. Manual or electric analysis of the spectrometer output will indicate a “hit” if certain predetermined output parameters are met. Colorimetric cholinesterase inhibition reaction chemistry is conducted on a second substrate portion using suitable reaction chemistry to generate optical color changes indicative of the presence or absence of a cholinesterase inhibitor. Analysis by visible spectroscopy at a suitable wavelength of the reaction products, either in solution or on a solid substrate, provides output that is optically analyzed, manually or by electronic means, to indicate the presence or absence of cholinesterase inhibitors, i.e., a “hit”. Simultaneous “hits” by both analysis methods are interpreted as a positive indication of the presence of a cholinesterase inhibitor in the original aerosol sample. A third portion of the deposited aerosol may be collected as an archive sample for later analysis by any suitable method. Preferably, the data manager and signal output provides for near-real time analysis of samples, with rapid processing of under 5 minutes. The dual results from the color sensor and IMS ensure a high degree of specificity while limiting the probability of a false positive response.

Additional objects, features and advantages of the present invention will become more readily apparent from the following detailed description of a preferred embodiment when taken in conjunction with the drawings wherein like reference numerals refer to corresponding parts in the several views.

### BRIEF DESCRIPTION OF THE DRAWINGS

FIG. 1 is a schematic representation of the apparatus of the present invention;

FIG. 2 is an IMS display graph for a 50 ng sample of the toxin simulant DFP;

FIG. 3 is an IMS display graph for a 200 ng sample of the toxin simulant methamidophos;

FIG. 4 illustrates the enzymatic rate of reaction for DFP and the enzyme AChE in the presence of the pH indicator Phenol Red;

FIG. 5 is an IMS display graph of a white candidate substrate material exposed to HTMI and AChE;

FIG. 6 is an IMS display graph of a white candidate substrate material exposed to a blank sample;

FIG. 7 is an IMS display graph of a tan candidate substrate material exposed to HTMI and AChE;

FIG. 8 is an IMS display graph of a tan candidate substrate material exposed to a blank sample;

FIG. 9 is a schematic depicting some common collector decision elements; and

FIG. 10 is a schematic of an apparatus of the present invention including a reel-to-reel sample distributing system.

### DETAILED DESCRIPTION OF THE PREFERRED EMBODIMENT

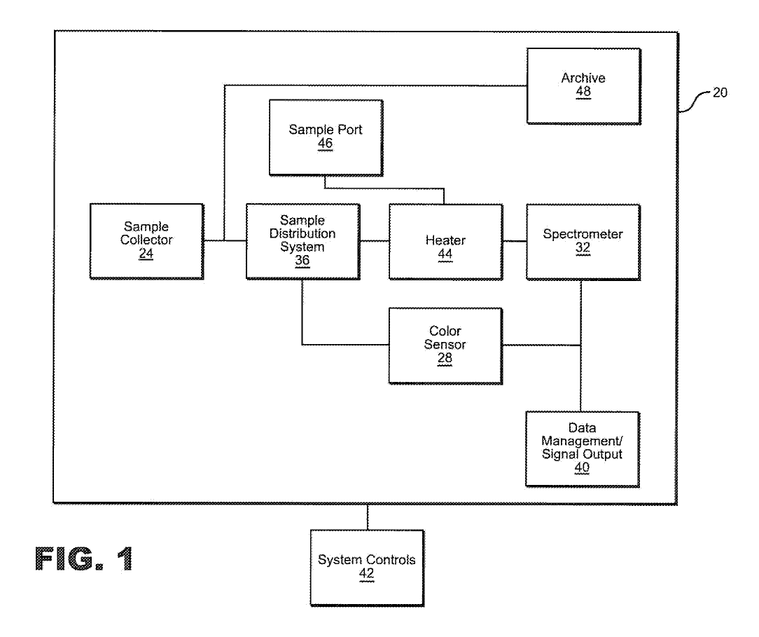

With initial reference to FIG. 1, a near-real time HTM detection system of the present invention is generally indicated at 20. System 20 is an orthogonal detection system (i.e., system based on dissimilar detection principles) for analyzing environmental samples including plural detectors in parallel or in series to increase the probability of HTM detection and decreases false positives. More specifically, detection system 20 includes a sample concentrator or collector 24, a color sensor 28 (e.g. colorimeter or spectrophotometer), an ion mobility spectrometer (IMS) 32, a sample distributing system indicated at 36 and a data manager and signal output 40. Detection system 20 also includes various system controls indicated at 42. A heater 44 is in fluid communication with spectrometer 32, and is also in communication with a sample port 46 adapted to receive swab samples or the like not collected through collector 24. Additionally, detection system 20 preferably includes an archive portion 48 adapted to archive samples for possible analysis at a later time. Detection system 20 may include a dry or wet cyclone collector, or any other collector capable of collecting liquid or solid aerosols, in either a batch or continuous mode, and concentrate them on a sample substrate.

Collector 24 concentrates and deposits aerosols, either in a dry or wet form, by impaction, filtration, or other suitable method, onto a substrate which may be a cloth, a membrane, or other suitable surface including test strips suitable for later analysis steps. Heater 44 is utilized to heat part of the deposited aerosol on the substrate by contact, convection or radiant heat to form a vapor that is then introduced into spectrometer 32. Among the materials to be detected by the present invention are low-volatility, cholinesterase inhibiting toxic materials. A detection limit equivalent to that of the IDLH (Immediately Dangerous to Live or Health) concentration for VX (i.e., 0.003 mg/m³) is preferred. Manual or electric analysis of the spectrometer output will indicate a “hit” if certain predetermined output parameters (such as drift time, peak shape, peak ratio compared to a calibrant peak or other parameters) are met. Another part of the deposited aerosol is transferred to a reaction vessel or substrate, or reacted in place, where colorimetric cholinesterase inhibition reaction chemistry is conducted using suitable reaction chemistry to generate optical color changes indicative of the presence or absence of a cholinesterase inhibitor. Analysis by visible spectroscopy (color sensor 28) at a suitable wavelength of the reaction products, either in solution or on a solid substrate, provides output that is optically analyzed, manually or by electronic means, to indicate the presence or absence of cholinesterase inhibitors, i.e., a “hit”. Simultaneous “hits” by both analysis methods are interpreted as a positive indication of the presence of a cholinesterase inhibitor in the original aerosol sample. A third portion of the deposited aerosol may be collected as an archive sample at archive portion 48 for later analysis by any suitable method. Preferably, data manager and signal output 40 provides an electronic and data management system allowing for near-real time analysis of samples, with rapid processing of under 5 minutes. The dual results from color sensor 28 and spectrometer 32 ensure a high degree of specificity while limiting the probability of a false positive response.

Prior to describing additional details of the preferred embodiment of the invention, experimental information associated with the development of the invention will be set forth, basically for the sake of completeness and further understanding of the overall invention.

### Experimental Procedures

### I. Test Design

A. Ion Mobility Spectroscopy

Initial trials were performed to check the response of the IMS to HTM simulants. The simulants tested were ethyl parathion, diisopropylfluorophosphate (DFP) and methamidophos. The simulants were prepared at varying concentrations in either methanol or pentane and tested on the IMS to evaluate its response, sample carryover and sensitivity.

B. Colorimetric/Enzymatic

The toxic effect of HTM nerve agents depends on the substance inhibiting the enzyme acetylcholinesterase (AChE) in the cholinergic nerve system. AChE is responsible for breaking down the signal substance acetylcholine, a process requiring two steps, i.e., acetylation by means of a serine in the active site and hydrolysis of the resulting acetylated enzyme.

Enzyme-OH+CH₃C(═O)—O—(CH₂)₂—N⁺(CH₃)₃ reacts with the release of choline to give Enzyme-O—C(═O)—CH₃ which is rapidly hydrolyzed to Enzyme-OH+CH₃COOH.

Degradation of acetylcholine in the cholinergic synapse takes place rapidly because the enzyme is available in large amounts and is extremely is active. Under optimum conditions, each enzyme molecule hydrolyzes about 15,000 acetylcholine molecules per second. The reaction of the enzyme with nerve agents is similar, but with the important difference that the rate of the final hydrolysis step is negligible. Consequently, the enzyme becomes irreversibly inhibited, with the nerve agent covalently bound to the enzyme via the serine in the active site.

Enzyme-OH—X—P(═O)(R₁)(—OR₂) releases HX to give Enzyme-O—P(═O)(R₁)(—OR₂).

Inhibition of AChE by a nerve agent is thus a cumulative process and the degree of inhibition depends not only on the concentration of nerve agent but also on the time of exposure. Traditional nerve agents are potent inhibitors of AChE. For example, a Soman concentration of 10⁻⁹ M is sufficient to inhibit the enzyme by more than 50% within 10 minutes.

Two methods of enzymatic colorimetric detection were evaluated, and are based on two different aspects of the reaction of cholinesterases with analytes of interest. The first of these methods is based upon the change in pH that results from the release of a hydrogen halide (HX) upon reaction of an analyte with cholinesterase. An added pH indicator allows colorimetric detection of this change. The second method is based on the inhibition of AChE by HTM nerve agents. In this test, AChE reacts with the substrate acetylthiocholine iodide (ATCI) to form a free sulfhydryl group. This sulfhydryl group reacts with the indicator 5,5′-dithio-bis-(2-nitrobenzoic acid) (DTNB) to give a yellow color that is detected by a spectrophotometer. When HTM is added to the matrix, some of the AChE is inhibited and reaction with ATCI is correspondingly inhibited. The amount of free sulfhydryl group is thus less and the change in the color intensity measured by the spectrophotometer is also less. The more HTM that is added, the more AChE is inhibited and the lower the intensity of the resulting color.

Initial trials were performed to check the response of the enzymatic test to the simulants DFP, ethyl parathion, methamidophos, and HTM1. The goal of these tests was to find optimum concentrations and reaction times for the enzyme, substrate and indicator. To optimize the sensitivity of the colorimetric reaction, various concentrations of the simulants, HTM1, enzymes, substrates, and indicators were tested. The color changes of the reaction solutions were measured via a spectrophotometer at the optimum wavelength for each color indicator tested.

### II. Test Procedures

A. Ion Mobility Spectroscopy

Standards were prepared for DFP, ethyl parathion and methamidophos in either methanol or pentane. Each standard solution (one simulant at a time) was spiked onto a clean swab and the solvent was allowed to evaporate (1 minute). Once the solvent had evaporated the swab was placed into the IMS for testing. Several solvents were tested by spiking the solvents onto a clean swab and reading the swab on the IMS after the solvent had evaporated. Solvents tested were dichloromethane (DCM), chloroform, acetone, methanol and pentane.

B. Colorimetric/Enzymatic

The enzymatic colorimetric tests were carried out as follows. A sample matrix of 4 mL deionized water and 1 mL Tris buffer was placed in a spectrophotometer sample cell in a temperature-controlled chamber at 37° C. and treated as follows. An aliquot of HTM was added to the matrix (sample). Then, 25 μL of AChE solution was added and the sample was allowed to react for 10 minutes. Next was added 25 μL of ATCI followed by 25 μL of DTNB, with the sample allowed to react 0.5 minutes after each addition. The sample was analyzed by a spectrophotometer after the final addition, and at 30 second intervals thereafter for a total of 5 minutes. The wavelength monitored was based on the indicator used. Although this is a qualitative test, a determination of the sensitivity and stability of the reaction was performed.

Individual standards were prepared for each of DFP, ethyl parathion, methamidophos and HTML. Each standard solution (one chemical at a time) was spiked in 4 mL of deionized water (Milli-Q). The amount of water was changed to 5 mL when the volume of reagents was changed. Several variants of this procedure were tested in order to optimize performance as a function of the reagents used. Several buffers were evaluated based on literature references. Specifically, Tris (tris(hydroxmethyl)aminoethane), MES sodium salt (4-morpholine-ethanesulfonic acid sodium salt), 2-(N-morpholino)ethanesulfonic acid sodium salt, MES hydrate (2-(N-morpholino) ethanesulfonic acid hydrate), 4-morpholine-ethanesulfonic acid, and HEPES (N-(2-Hydroxethyl)piperazine-N′-(2-ethanesulfonic acid)) were all tested for pH and reagent stability.

The enzymes evaluated were equine butyrylcholinesterase (BChE) and human AChE. Various concentrations of the enzyme were tested to find an optimized enzyme concentration. The substrates evaluated were butyrylcholine iodide and butyrylthiocholine iodide for the BChE and ATCI for the AChE. Concentrations of the substrates were maintained in excess. The indicators evaluated were Phenol Red (Phenolsulfone-phthalein sodium salt), Guinea Green (Aldrich part #207721), Malachite Green (4-N,N,N′,N′-tetramethyl-4,4′-diaminotriphenylcarbenium oxalate), and DTNB. Phenol Red is a pH indicator. Guinea Green, Malachite Green, and DTNB are based on the reaction of the enzyme with the substrate to form thiocholine, causing a change in the color of the indicator.

C. Enzyme Stability

In order to study the stability of the various reagents in the colorimetric/enzymatic detection system, the following experiments were carried out. ATCI was prepared in Tris buffer with the goal of making up the ATCI and DTNB in one solution. However, the background color of the ATCI added to the DTNB increased over time (1 to 2 days). A new working solution of ATCI was prepared and the mixture of the ATCI and DTNB was again clear. The next day the ATCI and DTNB was mixed and a pale yellow color was again observed. Another ATCI solution was prepared in deionized water and the new ATCI solution was mixed with DTNB on several days and no color was produced. Thus, ATCI is not stable in Tris buffer, and ATCI and DTNB will not be able to be prepared in a single solution.

Lyophilized powder of the AChE was purchased and diluted with 1 mL of deionized water. This stock solution was stored frozen. The working solution of the stock was made by diluting an aliquot of the stock with Tris buffer and another working solution was prepared by dilution with deionized water. The two working solutions were compared spectrophotometrically, and the working solution prepared in Tris buffer had a higher absorbance. Because of the higher absorbance of the Tris buffer solution, other AChE working solutions were made using the Tris buffer instead of deionized water.

### III. Quality Control

A. Ion Mobility Spectroscopy

A blank swab was tested at the start of the day and between samples that caused the IMS to alarm. The verification sample was analyzed to check the performance of the IMS. An IMS bake-out cycle was preformed when carryover to the blank sample was detected.

B. Colorimetric/Enzymatic

Reagent blanks were processed and measured along with the samples. A positive control samples was added that was measured before to and after each test.

### IV. Results and Discussion

A. Ion Mobility Spectroscopy

Ethyl parathion was the first simulant to be tested. Multiple peaks were observed. Attempts to improve detector response by changing the desorber temperature where unsuccessful due to the degradation temperature of ethyl parathion (120° C.). Thus, ethyl parathion was deemed unsuitable for testing purposes and replaced with methamidophos.

DFP was next tested, in both MeOH and pentane, to address possible interference from the MeOH. Two peaks were observed (DFP1 and DFP2), a common outcome in IMS spectra. The ratio of these two peaks was concentration dependent. Table 1 shows peak area data for DFP2, which was found to behave more linearly as a function of concentration than DFP1.

TABLE 1
DFP2 calibration data for DFP in pentane.

| Std | Average | Amount | | |
|---|---|---|---|---|
| Concentration | Area | Found | | |
| 2.5 | 0 | 2.24 | 2.5-20 | |
| 5 | 14.2 | 5.84 | Slope | 3.948 |
| 10 | 27.4 | 9.91 | Intercept | −8.86087 |
| 20 | 71 | 20.23 | RSQ | 0.9918 |
| 50 | 133.7 | 36.11 | | |
| 100 | 176.8 | 47.03 | | |
| 200 | 239.9 | 63.01 | | |
| 500 | 252.6 | 66.23 | | |

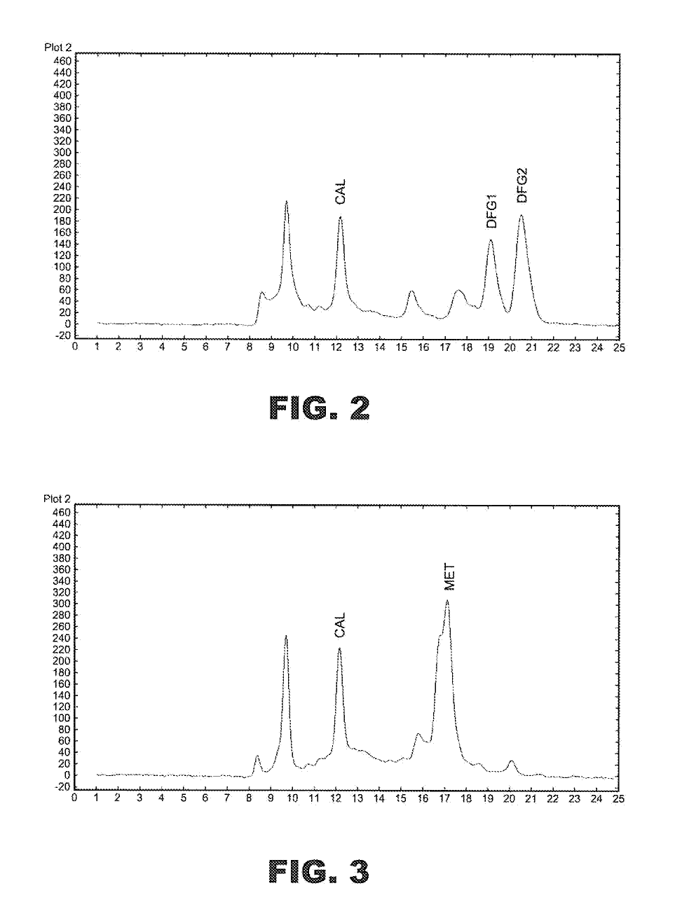

The range of sample sizes used for the calibration curve was 2.5 to 20 ng. Concentrations above 20 ng did not give a linear response. FIG. 2 is a screen capture of the IMS display of a 50 ng DFP sample. Different desorber, inlet and drift tube temperatures were tested to achieve the best response. The final optimized temperatures were 110° C. for the desorber, 110° C. for the inlet and 130° C. for the drift tube.

A third simulant, methamidophos, is insoluble in pentane, so was tested in MeOH. FIG. 3 shows a screen capture of the IMS display for a 200 ng sample of methamidophos. Different desorber, inlet and drift tube temperatures were tested to achieve the best response. The final optimized temperatures were 170° C. for the desorber, 200° C. for the inlet and 150° C. for the drift tube.

During the testing of methamidophos, issues related to sample carryover and response reproducibility were noted. Blank control tests after positive samples revealed a carryover problem. However, this may be related to the fact that the drift tube temperature of 150° C. was limited by the instrument control software. Because of the low volatility of methamidophos, we hypothesize that the carryover issue will be resolvable by attaining higher drift tube temperatures. In addition, it was observed that the peak areas were different if a new swab was used. An experiment was carried out to understand these differences. As shown in Table 2, the first measurement with a fresh swab is significantly different than subsequent measurements. This suggests that conditioning the swab to will improve the reproducibility of the measurement. An FTIR/Microscope analysis of the swab material'was completed and the swabs used in this testing were determined to be a Rayon material.

TABLE 2
Reproducibility of methamidophos IMS data.

| Replicate | Area |
|---|---|
| 1 | 118 |
| 2 | 46.9 |
| 3 | 59.9 |
| 4 | 67.1 |
| 5 | 43.5 |
| Average | 54.35 |
| Std Dev | 11.054 |
| % RSD | 20.3 |

A calibration curve was not calculated for methamidophos due to is the above carryover and reproducibility issues.

B. Colorimetric/Enzymatic

Two general detector reaction classes were explored: reactions generating a change in pH based on HTM reaction with cholinesterase, and reactions based on cholinesterase inhibition by reaction with HTMs.

### 1. pH Change

This method is based on the fact that hydrolysis of HTMs generate acidic species. HTMs based on phosphonates release phosphonic acids upon hydrolysis. In addition, many HTMs are phosphonyl fluorides, which additionally release hydrogen fluoride (HF) upon hydrolysis. This to is, in principle, a highly sensitive method for detecting HTMs, since the amount of HX that must be released to effect a pH change from neutral (pH 7) to an easily detectable change, i.e., approximately pH 6, is only 1×10⁻⁶ mol/L, corresponding to an HTM concentration on the order of 0.1 ppm.

Phenol Red is a pH indicator which changes color at pH 7.4. Since the expected hydrolysis reaction of HTM would result in a lowered pH, the detector system would have to be buffered to a pH higher than 7.4. However, it is anticipated that acidity generated as a result of HTM hydrolysis would be overwhelmed by the buffering capacity of the system, resulting in no color change of the indicator. Despite these difficulties, as shown in Table 3, the pesticides parathion and methamidophos were detected at levels varying from 300-500 ng and DFP and HTM1 were detected at levels varying from 25-50 ng.

Guinea Green and Malachite Green are expected to work at pH levels in the acid range. Malachite Green was prepared in a HEPES buffer (pH 5.4). Note that the Malachite Green must be maintained at a pH below 6.7 to avoid degradation and an attendant color change from blue to purple. In addition, Guinea Green is stable at a pH of 7.4, but the color fades at higher pH. A solution of Guinea Green was also prepared in HEPES buffer (pH 5.4).

Low pH buffers (pH 5.4) appear to adversely affect the enzyme and stopped the reaction. However, when 1 mL of Tris buffer (pH 7.8) was added to the solution to stabilize the enzyme, the indicators changed because of the buffer's effect on the pH of the solution. Because of these issues, we could not detect 625 ng of HTM1 using either the Guinea Green or Malachite Green indicators. Therefore, pH indicator detection systems were determined to be unsuitable for use with the present invention, and where not pursued further.

### 2. Cholinesterase Inhibition

The next technology tested was a mature colorimetric method for the detection of cholinesterase-inhibiting chemicals using a system comprised of AChE, ATCI, and DTNB. The ATCI and DTNB were prepared in Tris buffer (pH 7.8) and the AChE came in a potassium buffer. It was determined that ATCI and DTNB are light- and temperature-sensitive in solution. In addition, ATCI is not stable in the Tris buffer so it was prepared in water and a MES Hydrate buffer (pH 3.6). The ATCI appears to be stable in water if kept cold and in an amber vial. ATCI in MES buffer was found to cause problems with the enzyme.

A fresh enzyme stock solution was prepared in water and a dilution was made from the stock in water and another dilution was made in Tris buffer. The enzyme in the tris buffer solution has more activity than the enzyme prepared in water.

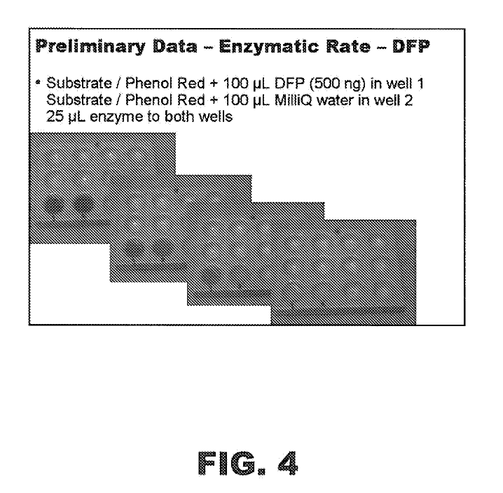

Using these techniques, 0.1 ng of HTM1 was detected using the DTNB, AChE and ATCI. The results for 1 ng are reproducible, with the 0.1 ng data having more scatter to them. We also studied methamidophos, parathion and DFP using the new solutions (AChE, DTNB, and ATCI) and could not detect 100 ng of methamidophos or parathion. We could not detect DPF at 25 ng. A summary of the results achieved using the enzymatic method is in Table 4 below. Typical to appearance is shown in FIG. 4.

TABLE 4
Summary of the enzymatic method results.

| Enzymatic | | Enzyme/Agent | Amount | | |
|---|---|---|---|---|---|
| Agent | Substrate | Time | Detected | | Comments |
| Phenol Red | | | | | Very narrow pH range |
| Parathion | BCI | 0.5 | 500,000 | ng | |
| Methamidophos | BCI | 0.5 | 290,000 | ng | |
| DFP | BCI | 0.5 | 50 | ng | |
| HTM1 | BCI | 0.5 | 25 | ng | |
| Guinea Green | | | | | Not stable in pH above 7.5 |
| DFP | BCI | 0.5 | >100 | ng | |
| HTM1 | ATCI | 5 | >2.5 | ng | |
| Malachite | | | | | Enzyme degrades in pH below 7 |
| Green | | | | | |
| HTM1 | BCI | 0.5 | >625 | ng | |
| | | | | | ATIC not stable in pH 7.8 |
| DTNB | | | | | Enzyme appears to have more |
| Parathion | ATCI | 0.5 | >100 | ng | activity if prepared in buffer |
| Methamidophos | ATCI | 0.5 | >100 | ng | (Tris pH 7.8) |
| DFP | ATCI | 0.5 | >25 | ng | |
| HTM1 | ATCI | 5 | ~0.1 | ng | |

### 3. Enzyme Stability

A study was then conducted on the stability of the AChE in Tris buffer. The AChE does not appear to be stable in Tris buffer. A working solution was prepared and split into 2 vials. Vial 1 was placed in a refrigerator and vial 2 was stored at room temperature. The AChE was reacted with ATCI and DTNB and reading were recorded from the spectrophotometer (HACH DR/2010) at 0.5 minute intervals for 5 minutes.

The procedure was as follows:
4 mL MilliQ water
1 mL Tris buffer
Zero spectrometer
Read control sample
Add 25 μL AChE mix
Add 25 μL ATCh mix and wait 0.5 minutes
Add 25 μL DTNB mix and wait 0.5 minutes
Read sample every 0.5 minutes for 5 minutes
Read control sample

Cold AChE, ATCI, and DTNB were kept on ice and in amber zo bottles during the day and in the refrigerator overnight. A room temperature (RT) AChE sample was stored in the hood at room temperature for the same period of time. On day 0 the cold and RT AChE samples had the same absorbance readings, but on day 1 the RT sample had more absorbance. The samples were tested again on day 4 and the difference between the stability samples was even larger. Both AChE solutions had lower absorbance than when solution was prepared, but the RT solution appears to have a slower rate of change. Table 5 below shows the difference of the initial absorbance reading from the 5 minute absorbance reading for the comparison. This tracks the rate change from day to day.

TABLE 5
Stability of the AChE solution (Difference absorbance reading).

| | Day | | | | | |
|---|---|---|---|---|---|---|
| | 0 | 1 | 4 | 0 | 1 | 4 |
| Test Date | Jul. 5, 2007 | Jul. 6, 2007 | Jul. 9, 2007 | Jul. 5, 2007 | Jul. 6, 2007 | Jul. 9, 2007 |
| Storage | Cold | Cold | Cold | RT | RT | RT |
| Temp | | | | | | |
| Stock | Jun. 12, 2007 | Jun. 12, 2007 | Jun. 12, 2007 | Jun. 12, 2007 | Jun. 12, 2007 | Jun. 12, 20077 |
| Dilution | Jul. 5, 2007 | Jul. 5, 2007 | Jul. 5, 2007 | Jul. 5, 2007 | Jul. 5, 2007 | Jul. 5, 2007 |
| Reading 1 | 0.884 | 0.513 | 0.330 | 0.841 | 0.746 | 0.543 |
| Reading 2 | 0.945 | 0.571 | 0.318 | 1.019 | 0.749 | 0.577 |
| Reading 3 | 0.809 | 0.493 | 0.319 | 0.844 | 0.668 | 0.544 |
| Reading 4 | | 0.422 | | | 0.644 | |
| Average | 0.879 | 0.500 | 0.322 | 0.901 | 0.702 | 0.555 |
| Std Dev | 0.0681 | 0.0615 | 0.0067 | 0.1019 | 0.0537 | 0.0193 |
| % RSD | 81.12 | 43.83 | 31.57 | 79.94 | 64.80 | 53.53 |
| % Difference | 0.0 | −38.0 | −55.7 | 0.0 | −20.0 | −34.7 |
| from day 0 | | | | | | |

Table 6 below shows the initial absorbance reading for the comparison.

TABLE 6
Stability of the AChE solution (initial absorbance reading).

| | Day | | | | | |
|---|---|---|---|---|---|---|
| | 0 | 1 | 4 | 0 | 1 | 4 |
| Test | Jul. 5, 2007 | Jul. 6, 2007 | Jul. 9, 2007 | Jul. 5, 2007 | Jul. 6, 2007 | Jul. 9, 2007 |
| Date | | | | | | |
| Storage | Cold | Cold | Cold | RT | RT | RT |
| Temp | | | | | | |
| Stock | Jun. 12, 2007 | Jun. 12, 2007 | Jun. 12, 2007 | Jun. 12, 2007 | Jun. 12, 2007 | Jun. 12, 2007 |
| Dilution | Jul. 5, 2007 | Jul. 5, 2007 | Jul. 5, 2007 | Jul. 5, 2007 | Jul. 5, 2007 | Jul. 5, 2007 |
| Reading 1 | 0.286 | 0.171 | 0.087 | 0.312 | 0.255 | 0.162 |
| Reading 2 | 0.294 | 0.184 | 0.089 | 0.331 | 0.238 | 0.164 |
| Reading 3 | 0.238 | 0.163 | 0.088 | 0.285 | 0.221 | 0.158 |
| Reading 4 | | 0.124 | | | 0.209 | |
| Average | 0.273 | 0.161 | 0.088 | 0.309 | 0.231 | 0.161 |
| Std Dev | 0.0303 | 0.0258 | 0.0010 | 0.0231 | 0.0201 | 0.0031 |
| % RSD | 24.24 | 13.47 | 8.70 | 28.62 | 21.07 | 15.83 |
| % Difference | 0.0 | −11.2 | −18.5 | 0.0 | −7.9 | −14.8 |
| from day 0 | | | | | | |

The temperature of the refrigerator used to store the cold sample was cycling between −16° C. and 2° C. so a new solution of AChE was prepared and stored in a refrigerator with a more stable temperature. The difference of the initial absorbance reading from the 5 minute absorbance reading was used for the comparison for the new AChE solution with the RT solution. RT data for days 5 and 6, as well as data for the new AChE to solution, are shown in Table 7 below.

TABLE 7
Stability of AChE stored at room temperature and a fresh cold
solution.

| | Day | | | | |
|---|---|---|---|---|---|
| | 5 | 6 | 0 | 1 | 2 |
| Test Date | Jul. 10,2007 | Jul. 11, 2007 | Jul. 9, 2007 | Jul. 10, 2007 | Jul. 11,2007 |
| Storage Temp | RT | RT | New | New | New |
| Stock | Jun. 12,2007 | Jun. 12,2007 | Jun. 12,2007 | Jun. 12,2007 | Jun. 12,2007 |
| Dilution | Jul. 5, 2007 | Jul. 5, 2007 | Jul. 9, 2007 | Jul. 9, 2007 | Jul. 9, 2007 |
| Reading 1 | 0.430 | 0.374 | 0.958 | 0.632 | 0.423 |
| Reading 2 | 0.432 | 0.369 | 1.089 | 0.501 | 0.407 |
| Reading 3 | 0.345 | 0.365 | 0.941 | 0.635 | 0.402 |
| Reading 4 | | | | | |
| Average | 0.402 | 0.369 | 0.996 | 0.589 | 0.411 |
| Std Dev | 0.0497 | 0.0045 | 0.0810 | 0.0765 | 0.0110 |
| % RSD | 35.27 | 36.48 | 91.50 | 51.28 | 39.97 |
| % Difference | −49.9 | −53.2 | 0.0 | −40.7 | −58.5 |
| from day 0 | | | | | |

A new supply of AChE was ordered but the only available AChE was in solution. The solution from the supplier is made up in HEPES buffer at pH 8.0. New working stock solutions in HEPES will need to be similarly tested for stability. These results show that there may be challenges associated with the various reagents and solutions required for this method. For example, ATCI and DTNB will have to be in a separate solution with the ATCI made up in water. The AChE is not stable in a to Tris buffer solution. Additional buffers that prevent degradation of enzyme are useful.

### V. Conclusions

Based on the foregoing experimental work, the following conclusions regarding the down-selected methods of detection are made.

A. Ion Mobility Spectroscopy

DFP was detectable by IMS, and at least one of the observed signals could be used to generate a very linear calibration curve. Ethyl parathion could not be detected by this method because of thermal instability. HTM1 was not tested on the IMS, but methamidophos, which has a similar vapor pressure, was detected by the IMS. Lack of to reproducibility of the signal precluded generation of a calibration curve for methamidophos. The simulants tested did not interfere with each other.

B. Colorimetric/Enzymatic

HTM1 can be detected at 0.1 ng using the AChE, ATCI, and DTNB reagents. The reaction time of 5 minutes when the chemical and the enzyme are mixed has better reproducibility than a 0.5 minute or 2 minute mixing time. The other indicators evaluated could only detect HTM1 at the higher level of 25 ng. The simulants used in this test would not cause a false reading unless the simulant concentration was above 50 ng. This is consistent with the weaker cholinesterase binding strength of the simulants.

The enzymatic system will not function properly if bleach vapors are present at the time of the test. The DTNB works on a lack of color if an “agent” is present, so if no “agent” is present a yellow color appears. But if bleach is present, the bleach can cause the color to fade for a false positive. A robust, fieldable version of the present invention will require a stable cholinesterase enzyme.

### VI. System Design and Method Optimization

A. Spectrophotometer

The HACH DR 2010 spectrophotometer that was used in these tests requires 5 mL of solution. However, the field detector will not be able to use that volume of liquid such that another type of spectrophotometer was tested. Morespecifically, an Ocean Optics (Spectrometer HR4000) detector was selected, particularly because of its ability to work in a reflection mode. Several experiments were completed to test if this type of spectrophotometer could be used. The reagents used were AChE, ATCI, and DTNB. The first experiment was to see if the dilution had an effect on the reaction. This was tested using a spot well plate.

The experiment conditions were as follows:
Store dark spectrum
Store reference spectrum
Add 25 μL AChE
Add 25 μL ATCI
Add 25 μL DTNB
Read sample in Absorbance mode, Transmission mode, and Reflection mode.

The graph produced was of the full spectrum, so a trend analysis in the strip chart mode was conducted in order to more easily view a difference at 412 nm. A difference could be observed between a blank well and the well with the three reagents. The color change was also visible without the detector.

The next experiment was to see the difference between the three reagents and the three reagents with the addition of HTM1. The procedure was the same as listed above with the addition of 5 μL HTM1 to the spot well plate before the addition of AChE. All of the spot well plate work was tested at room temperature. There was difference between the blank and the sample with the HTM1 added which was detected by the detector in the strip chart mode and by visual inspection.

Tests of the reagents on solid surfaces were also conducted. Filter paper and a cotton/polyester material were tried first. The liquid wicked on both types of material but both the detector and a visual observation could detect a change when the three reagents were added. The volume of reagents was changed from 25 μL to 5 μL to see if the spot could be more concentrated before HTM1 was added. This resulted in lighter spots when HTM1 was added. Both the detector and visual observation detected the difference. These data show that the Ocean Optics detector can be used to quantitate the difference in color on solid material.

B. Substrate Material

Several candidate materials were evaluated as reaction substrates. A tan-colored material (characterized in another program) that is ˜3.25 mm thick and a white-colored material ˜3.0 mm thick were tested using both the enzymatic system and the IMS. The enzymatic system was tested first. With the tan substrate, it was difficult to visually observe the color change and the spectrophotometer detector also detected only a slight change. On the white material, it was easier to visually observe the color change, and the detector had a larger absorbance change from the background. The addition of the reagents appeared to form a smaller spot than on filter paper and the cotton/polyester substrate, but they did spread over time. HTM1 was only added to the white material and there was a difference detected compared to a reagents-only spot. HTM1 and AChE were allowed to react for 5 minutes in the temperature control chamber before the addition of the ATCI and DTNB.

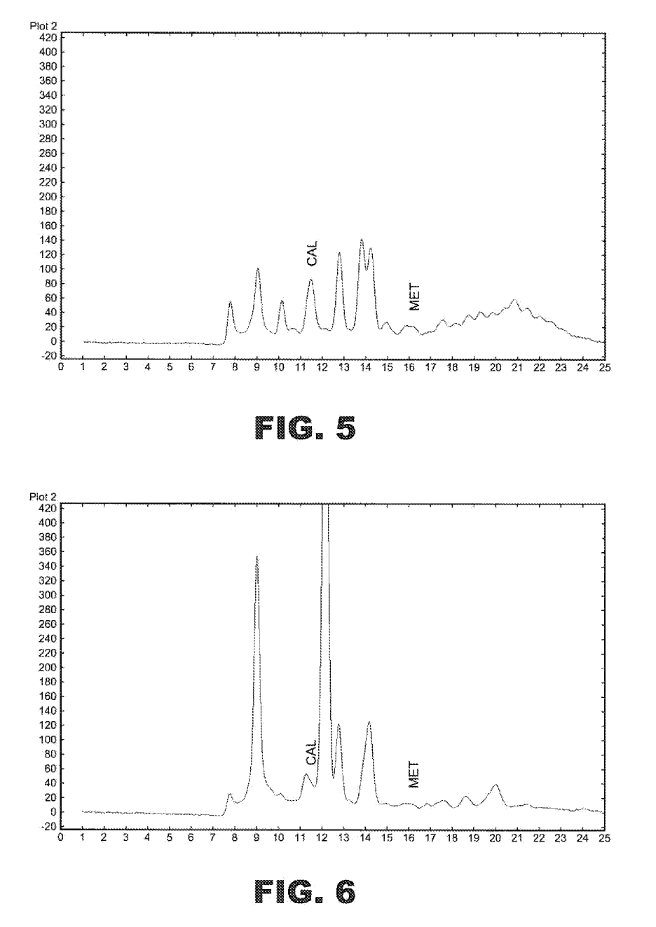

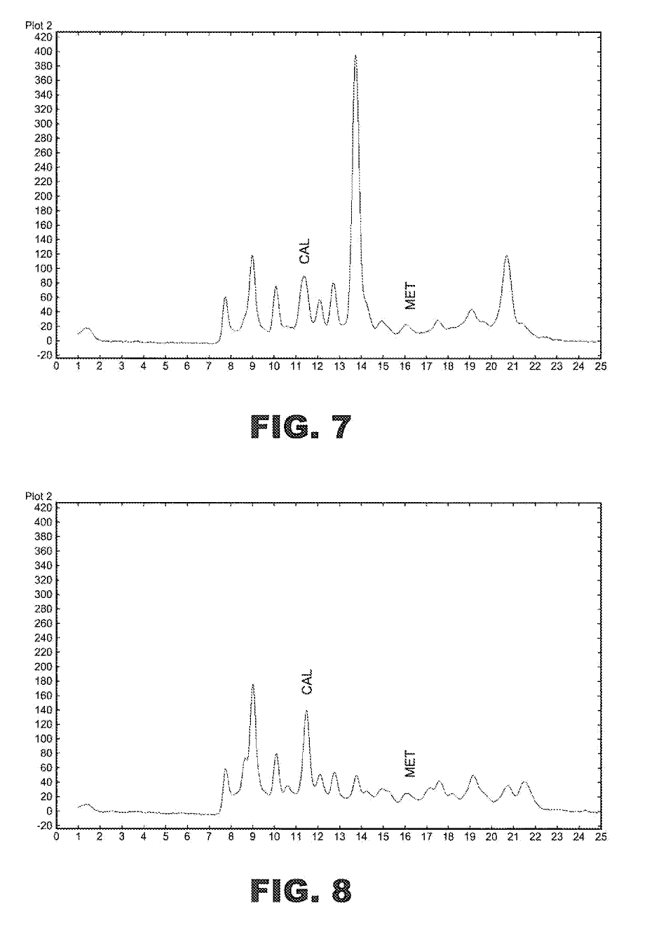

Both of the candidate materials tested on the IMS had peaks that could reduce the response of the IMS because of calibrant reduction, could interfere with the HTM1 peak, and could cause carry over. The is following plots are from the white and tan candidate material and blank swabs after the candidate materials. Seven blank swabs were run after the white candidate material before the tan candidate material was tested. FIG. 5 shows a sample scan of the white candidate material, and FIG. 6 shows a scan of a blank after the white candidate material. FIGS. 7 and 8 show similar scans for a tan candidate material.

A swab was also tested that had been spiked with 5 μL AChE to test for interference. After that swab was tested 10 μL of AChE was added and tested. ATCI was then added to the swab and tested. There were peaks found after the ATCI was added that could reduce the response of the IMS because of calibrant reduction, interfere with the HTM1 peak, and cause carry over.

C. Cholinesterase Stabilization

AChE (EC 3.1.1.7) is an efficient eukaryotic and plant-derived serine hydrolase enzyme that catalyzes the hydrolysis of acetylcholine to choline at rates that are nearly diffusion-limited. AChE is readily purified from various organisms and tissue subtypes. AChE gene sequences have been cloned for several derivatives, and functional enzymes can be produced from both native and mutagenized protein expression systems.

The AChE active-site catalytic triad is competitively and irreversibly inhibited by organophosphate (OP) compounds and carbamates. This potent inhibition can be leveraged for the sensitive detection of environmental neurotoxins. While AChE-based biosensors offer a rapid and sensitive interface for OP detection, enzyme stability limits many potential applications. It has been shown through functional analysis that AChE (Torpedo californica) loses activity within minutes at is temperatures greater than 35° C., and global structure is lost above 56° C. The authors of this work did not implicate a precise mechanism for loss of activity. However, global AChE conformational stability and thermal inactivation parameters were determined. Other work suggested that surface-exposed aromatic amino acids supported thermal-induced conformational scrambling and loss of activity. Indeed, mutagenized recombinant AChE variants have since been produced where amino acid substitutions markedly impacted enzyme stability. Glycosylation-deficient mutants have likewise been produced to demonstrate the importance of post-translational modifications to net enzyme stability. This suggests that enzyme source (species, tissue subtype) and purification will impact long term stability as processing and glycosylation patterns are expected to be unique. It is known that the thermal transition temperature (Tm) for human AChE is higher than that of either Torpedo or Bungarus AChE. Both hydrophobic and redox-active residues have also been implicated in AChE stabilization. Perhaps most striking, co-purified contaminates in the enzyme preparation have been shown to potentiate degradation. Previous affinity purification strategies using procainamide as a reversible AChE binding agent have been shown to co-purify protease enzymes which resulted in efficient enzyme cleavage and inactivation in solution and as a function of temperature. These contaminates may originate from either recombinant or native sources. A custom purification strategy employing an affinity histidine tagging system followed by a procainamide affinity chromatography was developed to eliminate contaminating protease enzymes. Human AChE (Sigma) is listed as an “affinity purified” reagent, thus implying the use of procainamide or similar affinity resin.

Decay of enzymatic activity may originate from intrinsic structural transitions (e.g. deamidation, dealkylation, denaturation, hydrolysis) due to unfavorable environmental parameters such as heat or solvent properties or from contaminate-driven chemical modifications of the holoenzyme complex (e.g. oxidation, phosphorylation, proteolysis) leading to activity modulation or degradation. Thermal-induced inactivation of purified AChE has been shown to proceed through a rate-limiting partial or local destabilization event which potentiates hydrophobic stacking through a molten globule state, global denaturation to an unfolded state, and concomitant aggregation or hydrolysis. Water is key to conformational flexibility and structural transitions such as deamidation and hydrolysis of peptide bonds. Hydration of the enzyme microenvironment, as well as surface electrostatic and hydrophobic properties are important factors to consider in the general design of stabilizing formulations for purified enzyme preparations. In fact, non-aqueous catalysis is an emerging paradigm for commercial and industrial-scale enzyme applications.

Recent efforts have been made to extend the shelf-life of AChE preparations using rational mutagenesis, solution formulations, and solid-phase encapsulation approaches. Encapsulation matrices have included hydrogels, synthetic polymers, mesoporous silica, liposomes, and nanocomposites. Varying degrees of stabilization for encapsulated and lyophilized AChEs at ambient temperatures have been noted, but only polyurethane encapsulating foams have shown marked stability enhancements at elevated temperatures. Similar, but less dramatic stabilization has been demonstrated with silica-based encapsulants. Implementation, transducer interface, and transport-limited diffusion of is the analyte should be considered in biosensor design with encapsulated enzyme systems.

Solution stabilization is a complex process that can be approached by high throughput combinatorial screening of stabilizing excipients using a rational selection strategy for a desired chemical property. Classes of excipients include antioxidants, surfactants, amino acids, oligosaccharides, and hydrating polymers which act non-covalently to stabilize the native enzyme structure or raise the free energy of the molten globule intermediate thereby altering the folding reaction equilibrium. ABAT has recently succeeded in applying this combinatorial screening strategy for the improved shelf-life of an important commercial enzyme. Because every protein is unique in structure and function, this process must be rationally designed and optimized accordingly.

Several stabilizing excipients have recently been described for AChE. These include choline chloride, sodium acetate, acetylcholine iodide, glycerol, sucrose, exogenous proteins, polyethylene glycol, and various divalent cations. This data could serve as the basis for an expanded combinatorial screen for AChE stabilization at ambient and elevated temperatures. Optimized formulations could be used to stability test AChE stored in solution and as a lyophilized pellet. A commercially available lyophilized AChE is quoted as stable for 6 months at 37° C. (Applied Enzyme Technology) and there are open source references to support this and other cryopreservation strategies, provided enzymatic activity is unaltered during the drying process.

Experimental assessment of enzyme activity may be achieved directly by functional assay using epitope-specific ELISA or substrate conversion or indirectly by following structural transitions using various analytical tools such as FT-IR, Raman spectroscopy, circular dichroism, quartz crystal microbalance, or differential scanning calorimetry. Enzyme integrity can be measured using chromatography, electrophoresis, and mass spectrometry. Both direct and indirect methods provide levels of detail useful in identifying destabilizing and stabilizing parameters.

Based on current knowledge, the present invention preferably utilizes cholinesterase stabilized using one or more of the methods set forth below. Varying degrees of AChE stabilization have been achieved through recombinant mutagenesis, solid-phase encapsulation, and through added excipients, although no full scale combinatorial stability screening has been described. Source and purity is known to impact stability and modified purification methods have been developed as discussed above. ABAT has demonstrated expertise in recombinant genetics, protein purification, and rational design of high throughput stabilizing formulations.

A detection device concept-of-operations is an important driver for determining the most appropriate stabilization strategy. When considering a reservoir of enzyme reagent that is delivered to a spotted sample or a vacuum sealed multi-well plate pre-loaded with enzyme, the latter scenario likely offers greater potential for long term stability against thermal fluctuations. A commercially available lyophilized product could be used to benchmark stability improvements in the formulation process. An AChE clone would provide a cost-effective and renewable source of product, molecular capabilities for stabilizing mutant formulations, and access to recombinant strategies for affinity purification. Affinity purified product could then be combinatorially screened for stabilizing excipients or encapsulants. Stability of the lead formulations could be temporally monitored at varied temperatures in dried or hydrated conditions. The resultant product is envisioned to afford seamless implementation to a deployable device with known tolerances and maintenance schedule.

D. Sample Collection Considerations

The front end of all detection and identification systems is a collector that can remove the material from the air and concentrate the material into a dry or liquid sample that is compatible with the detection or identification technology. In the case of a low-volatility HTM detection system, it is assumed that the HTM in the air will exist primarily as an aerosol and not a vapor. The aerosol may be a primary or secondary aerosol, i.e., it may consist of pure HTM in liquid or solid form, or as a liquid or solid deposit on a substrate aerosol particle. Therefore, an appropriate collector will more likely resemble a bioaerosol particle collector than a traditional chemical vapor collector. An aerosol collector evaluation was performed to identify preliminary collector requirements and potential collection mechanisms that could be applied to an HTM detection system.

Preferred attributes of the present invention are set forth below in order to aid in the choice of hardware for use with the present apparatus. In addition to the requirements related directly to the HTM properties, the requirements for a system collector will be dependent on the detection and identification systems chosen and their specific operational parameters and sample requirements. A list of preferred HTM collector requirements follows. Aspects of the requirements that are detector specific are noted.
General: The collector removes particles containing or consisting of HTM from the air and concentrates the material into a sample matrix compatible with the detection or identification method. (Current embodiment provides for collection onto a dry substrate).
Concentration: The ratio of HTM concentration in the sample to HTM concentration in the air must be determined and is related to detector sensitivity and the system sensitivity goal of 0.003 mg/m³. Sampling rate, collection efficiency and deposition area or volume may be adjusted as necessary to achieve the concentration goals.
Particle Size: The collector has a D₅₀ cut point less than or equal to 1 μm. There is no upper particle size restriction.
Contamination: The collector airflow path is designed to minimize loss due to potentially sticky particle collision with the walls of the collector.
Media Format: Collection onto a dry substrate is preferred. Sample handling is automatable. The sample is able to be delivered to multiple platforms (up to 3).
Desorption-Specific Requirements: The collector medium provides a sample capable of desorption into a detector (i.e., IMS). The substrate is resistant to heat at the desorption temperature required. The substrate does not interfere with the IMS spectral signature.
Colorimetric-Specific Requirements: The collector provides a sample compatible with colorimetric analysis. The substrate supports a platform for sample and chemistry reaction. The substrate does not interfere with the color-forming chemistry.
Confirmatory sample: The collection system provides a confirmatory sample.

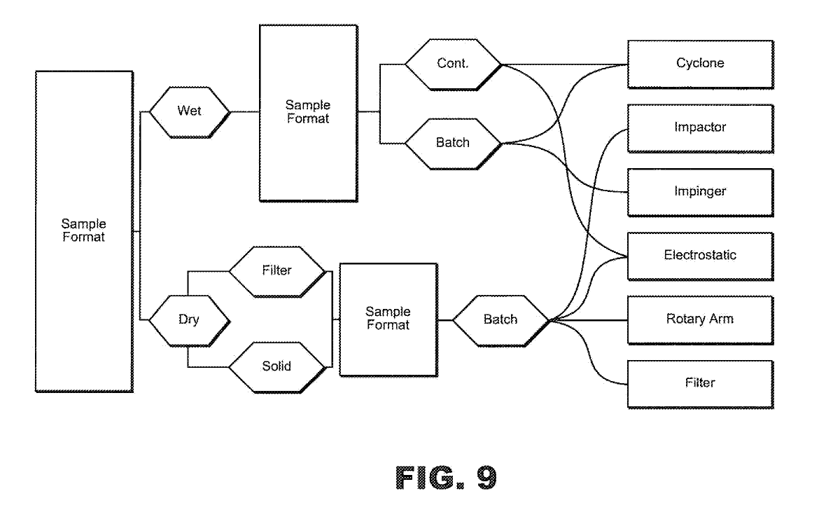

A collection overview was compiled to describe the types of aerosol collection mechanisms available and to aid in the down selection of collector technologies. The types of collectors can be categorized by sample format (wet vs. dry), operational modes (continuous vs. batch), particle deposition method (inertial vs. electrostatic), and performance parameters (high volume vs. low volume). Generally, the sample format will be defined by the sample analysis method, and mode of operation will be defined by the system requirements. Examples of some common collector decision elements are shown in FIG. 9.

Based on preliminary detection component down-selection to IMS and a colorimetric chemistry approach, a collector for use with this system preferably includes the following: the current form of the IMS requires a dry sample that can be desorbed through the application of heat and delivered to the detector. The colorimetric approach is most readily done in a liquid sample, however it is possible to perform the chemistry in a dry substrate. Further, wet sampling adds, complication to the collection system, increases the logistics footprint, and may unnecessarily dilute the sample. Therefore, the preliminary collector concept will favor a dry collection technology. A dry filter material also allows mixing of the sample with the colorimetric chemistries more readily than a solid surface. Preferably, the collector will operate in batch mode, with the sample concentrated into the filter over a period of time prior to delivery to the detector. The particle deposition method will depend on the filter material selected and the sampling rate required to achieve the target system sensitivity. The two potential particle deposition methods are high volume filtration or high volume impaction.

Preferably, a collector using a dry filter substrate provides samples to the IMS, colorimetric detector, and a confirmatory sample simultaneously and automatically either by flowing the sampled air through the filter or by impacting airborne particles on the filter surface (to achieve higher sampling rates). Alternatively, a low volume filter could be used in conjunction with an air-to-air concentrator. However, HTM particle deposition in the pre-impactor would be a major concern.

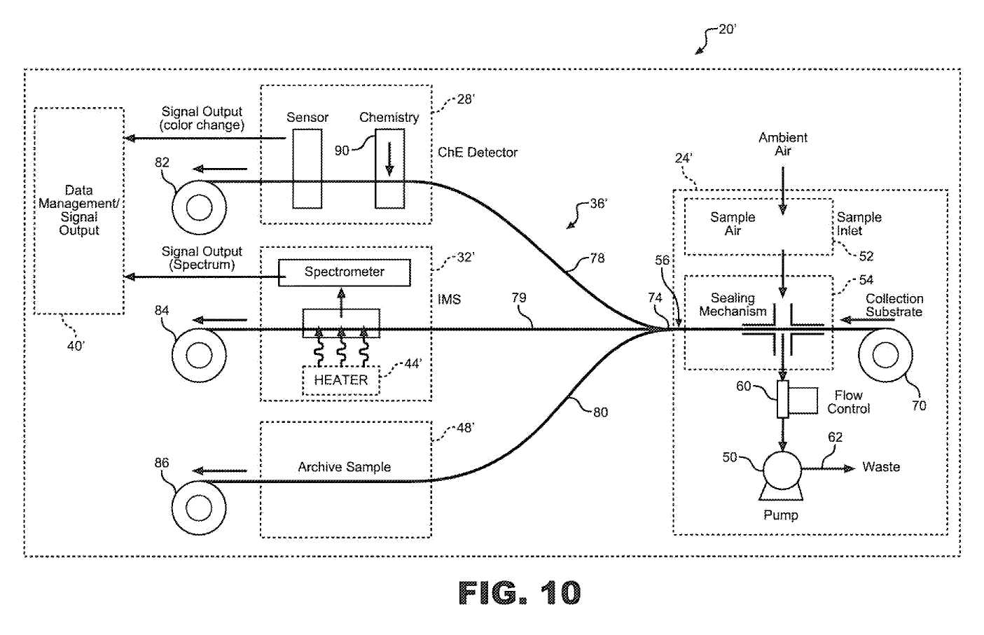

With the above-experimental data in mind, an automated detection system 20′ of the present invention was developed, which will now be discussed with reference to FIG. 10. As depicted, detection apparatus 20′ includes a collector 24′, a color sensor 28′, a reel-to-reel distributing system 36′ and a data merger and signal output 40′. An air pump 50 is adapted to draw ambient air through a sample inlet 52 in sample collector 24′ and directs the ambient air to a sealed sample concentrating portion 54. Preferably, sample collector 24′ is in the form of a high-volume aerosol collector. Pump 50 causes ambient air to impinge on a sample collection substrate 56, wherein any HTM particles in the air are collected by and concentrated on substrate 56. Airflow continues past substrate 56 through a flow controller 60 and out a waste port indicated at 62. In the embodiment shown, substrate 56 is a cloth or membrane tape which distributes concentrated samples simultaneously and automatically to color sensor 28′ and spectrometer 32′ having an associated heater 44′. Preferably, system 20′ also includes an archive sample portion 48′, which also receives a concentrated sample and archives the sample for potential further analysis at a later time.

The reel-to-reel system includes a first reel 70 located in sample collector 24′ which feeds a plurality of substrate layers indicated at 74 to sample concentrating portion 54. Substrate layers 74 exit concentrating portion 54, and first, second and third layers 78, 79 and 80 are fed to separate areas of detecting system 20′. More specifically, first layer 78 is attached to a second reel 82 such that first layer 78 and any HTM's thereon can be fed through color sensor 28′; second layer 79 is attached to a third reel 84 such that second layer 79 and any HTM's thereon can be fed through spectrometer 32′; and third layer 80 is attached to a fourth reel 86 such that third layer 80 and any HTM's thereon can be fed through archive sample portion 48′. A reagent applicator may be utilized to apply the reagents ATCI, DTNB and AChE to layered substrate 56 or to individual substrate layers 78-80 either before sample collection/concentration or before analysis. In the embodiment shown, color sensor 28′ includes a reagent applicator 90 for applying one or more of the reagents ATCI, DTNB and AChE to substrate 78. The same reagents (i.e. ATCI, DTNB and AChE) are utilized for ion spectroscopy. Results of the analysis can be determined utilizing data management/signal output unit 40′. Although not shown, it is contemplated that additional substrate layers could be fed to other detectors.

Selection of the appropriate substrate material is an important link between the operation of the collection portion and the color sensor and spectrometer devices. An ideal substrate allows a highly concentrated sample to be collected (high sampling rate and/or collection efficiency), is compatible with the detection processes (e.g. temperature, chemical, desorption and signature compatibility), and is thin enough to allow long duration reel-to-reel operation in a reasonably small space. A substrate material commonly used in a dry filter sampling application is a polyester felt material (PEF). The PEF filter has been shown to provide a high collection efficiency while allowing high sampling rates (˜500 LPM through a 47 mm diameter filter). However, PEF filter has limitations on the upper temperature range and may be substituted with Nomex® felt material when high temperature operation is required. Both PEF and Nomex® are thick (˜4 mm) and may be problematic to install in the reel-to-reel system. Therefore, a thinner material that can retain the filtered or impacted particles and allow proper distribution of the colorimetric chemicals such as cotton flannel, rayon, or other fabrics may be used.

Although described with reference to a preferred embodiment of the invention, it should be readily understood that various changes and/or modifications can be made to the invention without departing from the spirit thereof. For instance, although discussed with reference to cholinesterase inhibitors specifically, it should be understood that the present invention can be utilized with other classes of chemical aerosols by substituting suitable colorimetric indicator technologies (including but not limited to enzyme selection) and operating the visible spectrometer and IMS under the appropriate conditions. Additionally, modifications to the invention may include integration with a third detector output for increased confidence, networking with multiple detectors of the same or different kind, pretreatment of the substrate with all or some of the is needed reagents, use of multiple reagent sets to detect more than one chemical class of HTM using the same collected sample, or miniaturization of the sampling or detecting components. In general, the invention is only intended to be limited by the scope of the following claims.

1. A toxic material detection apparatus comprising:
a sample collection portion including a sample inlet and a sample concentrator adapted to concentrate an environmental sample on a substrate;
a color sensor adapted to analyze the substrate for a presence of toxic materials;
a spectrometer separate and distinct from the color sensor and adapted to analyze the substrate for a presence of toxic materials;
a sample distributing system adapted to distribute the environmental sample to the color sensor and the spectrometer simultaneously; and
a data management unit in communication with the color sensor and spectrometer.

2. The apparatus of claim 1 further comprising: an archive portion, wherein the sample distributing system is adapted to distribute the environmental sample to both the color sensor and the spectrometer, as well as to the archive portion for possible later analysis.

3. The apparatus of claim 1, wherein the sample distributing system is in the form of a reel-to-reel system and the substrate is in the form of a substrate tape.

4. The apparatus of claim 3, wherein the distributing system includes a first reel of layered substrate material including first and second layers; a second reel in communication with the spectrometer and connected to the first layer of the substrate material; and a third reel in communication with the color detector sensor and connected to the second layer of the substrate material.

5. The detection system of claim 4, further comprising: an archive portion, wherein the sample distributing system further comprises a fourth reel in communication with the archive portion and connected to a third layer of the substrate material.

6. The apparatus of claim 3, wherein the substrate is constituted by a cloth or membrane.

7. The apparatus of claim 6, wherein the substrate is selected from the group consisting of a polyester felt, a Nomex felt or a fabric material.

8. The apparatus of claim 1, wherein the sample collection portion is a high volume aerosol collection system including a dry cyclone collector.

9. The apparatus of claim 8, wherein the collection system is adapted to operate in batch mode.

10. The apparatus of claim 9, wherein the substrate is in the form of a filter material.

11. The apparatus of claim 1, wherein the sample collection portion includes a pump adapted to draw ambient air into the sample inlet such that the ambient air impinges on the substrate.

12. The apparatus of claim 1, wherein the sample collection portion includes a wet cyclone collector.

13. The apparatus of claim 1, further comprising: a reagent applicator for applying one or more reagents to the substrate.

14. The apparatus of claim 1, wherein the substrate is a pretreated substrate including one or more reagents thereon.

15. The apparatus of claim 1, further comprising:
a sample port; and
a heater interposed between the sample port and the spectrometer.

16. The apparatus of claim 1, wherein the spectrometer is an ion mobility spectrometer.

17. A method for detecting a toxic material comprising:
impinging ambient air onto a substrate to concentrate any airborne aerosols on the substrate;
distributing a first portion of the substrate to a color sensor and simultaneously distributing a second portion of the substrate to a spectrometer which is separate and distinct from the color sensor;
detecting a presence of toxic materials on the first portion of the substrate utilizing the color sensor;
detecting a presence of toxic materials on the second portion of the substrate utilizing the spectrometer; and
determining whether toxic materials are present on the substrate based on both the presence of toxic materials detected utilizing the color sensor and the presence of toxic materials detected utilizing the spectrometer.

18. The method of claim 17, wherein determining the presence of toxic materials is conducted electronically.

19. The method of claim 17, wherein determining the presence of toxic materials is conducted manually.

20. The method of claim 17, wherein the color sensor detects the presence of toxic materials based on cholinesterase inhibition.

21. The method of claim 20, further comprising: adding the reagents acetylcholinesterase, 5,5′-dithio-bis-(2-nitrobenzoic acid) and acetylthiocholine iodide to the substrate prior to determining the presence of toxic materials on the substrate.

22. The method of claim 17, wherein the aerosols are deposited on the substrate through high volume filtration.

23. The method of claim 17, wherein the aerosols are deposited through high volume impaction.

24. The method of claim 17, further comprising: distributing a third portion of the substrate to an archive portion for possible later analysis.
